(19) United States
(12) Patent Application Publication
(10) Pub. No.: US 2008/0319398 A1
(43) Pub. Date: Dec. 25, 2008

(54) Catheter-to-Device Locking System

(75) Inventor: Raymond Bizup, Feasterville, PA (US)

Correspondence Address: Fox Rothschild LLP;Blue Bell, 2000 Market Street, Tenth Floor, Philadelphia, PA, 19103, US

(73) Assignee: Medical Components, Inc., Harleysville, PA (US)

(21) Appl. No.: 12/139,699

(22) Filed: Jun. 16, 2008

Related U.S. Application Data
(60) Provisional application No. 60/936,263, filed on Jun. 19, 2007.

(51) Int. Cl. A61M 5/32

(52) U.S. Cl. 604/175

(57) ABSTRACT

A locking component (100) for locking a catheter proximal end section (32) onto a stem (56) of a medical device such as a venous access port assembly (50). The locking component (100) includes a smaller diameter compression surface (106) along a larger diameter channel (108) therethrough. The device stem (56) includes at least one protuberance (58) of greater diameter than the inner diameter of the catheter end portion (32), and a smaller diameter stem section (66) proximally of the protuberance. Preferably, all protuberances 58) of the stem are rounded. When a stop section (112) of the locking component (100) abuts a corresponding stop section at the proximal end of the stem (56), the compression surface (106) is disposed over and around the smaller diameter stem section (66) and compresses the portion of the catheter proximal end section (32) interposed therebetween thus locking the catheter onto the stem.

### CROSS-REFERENCE TO RELATED APPLICATIONS

This application claims priority from U.S. Provisional Patent Application Ser. No. 60/936,263 filed Jun. 19, 2007.

### FIELD OF THE INVENTION

This relates to the field of medical devices and more particularly to catheter assemblies and ports therefor, for the infusion of fluids into the patient and withdrawal of fluids from the patient.

### BACKGROUND OF THE INVENTION

Infusion ports for the infusion and/or withdrawal of fluids from a patient are well-known, secured to the proximal end of an implanted catheter. These ports are typically used for drug infusion or small amounts of blood withdrawal, where large flows of fluid are not required. The ports are assemblies of a needle-impenetrable housing with a discharge port in fluid communication with the catheter and the reservoir within the port housing, and provide a subcutaneous self-sealing septum that defines an access site for multiple needle sticks through the covering skin tissue of the patient, through the septum and into the reservoir, without the need to continuously search for new access sites. Examples of such ports are disclosed, for example, in U.S. Pat. Nos. 4,704,103; 4,762,517; 4,778,452; 5,185,003; 5,213,574; 5,637,102 and 6,113,572.

U.S. patent application Ser. No. 11/724,945 filed Mar. 16, 2007 and partially assigned to the assignee hereof, discloses a venous access port having a housing and a needle-penetrable septum enabling infusion of fluid into the port, providing an interior reservoir and a passageway extending from the reservoir through a stem of a discharge port to establish fluid communication with a proximal end of a catheter lumen to which the port assembly is secured prior to placement of the assembly into a patient. The discharge port is defined by a discharge stem extending from the port assembly and is adapted to be inserted into the lumen of the proximal end of a catheter that extends into the vasculature of the patient. The discharge stem defines a pair of barbed protuberances onto which the catheter lumen wall grips tightly when the stem is fully inserted into the catheter proximal end. This is seen in FIGS. 1 to 4 hereof. FIGS. 3 and 4 depict generally the procedure of locking a catheter proximal end onto the discharge stem using a locking sleeve.

U.S. patent application Ser. No. 11/801,050 filed May 7, 2007 and assigned to the assignee hereof, discloses a venous access port having a housing and a septum, providing an interior reservoir and a passageway extending from the reservoir through a stem of a discharge port to establish fluid communication with a proximal end of a catheter lumen to which the port assembly is secured prior to placement of the assembly into a patient. The housing includes a base and a cap that together cooperate to secure a needle-penetrable septum within the assembly by compressing a seating flange of the septum in a seat of the housing base. The cap is mechanically secured to the housing base by a mechanical joint and solvent bonding. As in the port discussed above, the discharge port is defined by a discharge stem extending from the port assembly and is adapted to be inserted into the lumen of the proximal end of a catheter that extends into the vasculature of the patient. The discharge stem defines a pair of rounded protuberances onto which the catheter lumen wall grips tightly when the stem is fully inserted into the catheter proximal end.

It is important to provide a venous access port assembly that provides an assured locking connection between the discharge stem and the catheter. U.S. Pat. No. 6,113,572 sets forth a connection system between a catheter's proximal end and a venous access port assembly at a discharge stem thereof. The rigid, tubular stem has a plurality of engagement barbs encircling and radially outwardly extending on an exterior surface of the stem. A compression sleeve is loosely disposed along the catheter, and the catheter's proximal end is first urged onto and over the engagement barbs, and then the compression sleeve is urged from its loose position over the catheter end fitted onto the discharge stem thus lockingly and sealingly securing the catheter to the discharge stem, in one embodiment for one type of catheter made of silicone material. For a catheter of polyurethane material, a compression sleeve is fitted onto a length of tubing larger in inner diameter than the catheter's outer diameter; when the catheter is force-fit onto the barbed discharge stem, the sleeve/tubing component is moved proximally over the catheter end, compressing the catheter lockingly and sealingly onto the discharge stem. In both embodiments, the engagement barbs have sharply defined proximal edges at their end faces against which the catheter lumen wall is firmly pressed by the locking system. The two types of connection systems are sold together with the venous access port assembly to enable the practitioner to select the appropriate one depending on the catheter selected by the practitioner.

It is desired to provide a catheter/device connection system that does not press the catheter wall directly against sharp edges of engagement barbs.

### BRIEF SUMMARY OF THE INVENTION

The present invention provides a locking connection system between a catheter end and a medical device. Such system may be used with venous access port assemblies, but may also be utilized with other catheter-connecting devices such as luer fittings, hubs and tunnelers. With respect to such devices, generally a stem of each such device is force-fit into the proximal end of a flexible tube such as a catheter (or an extension tube for a catheter assembly). The present invention provides a locking component that is slid over the catheter proximal end after the catheter end has been urged over the stem. The locking component is adapted to minimize damage to the catheter lumen wall by the protuberances of the stem when the catheter lumen wall is compressed by the locking component against the stem and not compressed directly against the protuberances of the stem when in its fully locked position.

The locking component of the present invention is preferably a one-piece sleeve that is disposed about a catheter at the proximal end thereof and is slid along the catheter proximal end portion after the catheter proximal end has been urged onto the stem of a medical device. A compression surface along the channel of the locking component is positioned to be disposed over the end of the catheter past the second protuberance, to compress the catheter lumen wall against the stem axially offset proximally from the most proximal protuberance. The channel of the locking component preferably includes a stop section adjacent the proximal end of the locking component, that abuts against a shoulder at the proximal end of the stem thus positioning the compression surface proximally of and axially offset from the second protuberance. Preferably, the stop section is beveled and rounded to facilitate being urged over the catheter proximal end portion especially where the catheter lumen wall has been expanded to pass over the rounded protuberances of the port stem. Preferably, also, distally of the smaller diameter compression surface is a transition to a larger diameter channel remainder extending to the distal end of the locking component, and the transition may be a more sharply angled ledge to deter movement of the locking component in the distal direction away from the port thus maintaining the locking component in its desired axial position.

One advantage of the locking component of the present invention is that, in its fully locked position, it does not compress the catheter lumen wall against a protuberance of the stem, thus preventing damage to the catheter wall by the locking component. Another advantage is that the compression surface, as it passes over the one, two (or more) protuberances of the stem and temporarily compresses the catheter lumen wall thereat, serves as a tactile indicator to the practitioner that the first and subsequently the second (or additional) protuberances have been successfully cleared by the compression surface of the locking component, and is in its appropriate final locking position as the stop section abuts the shoulder of the stem, thus preventing the practitioner from stopping merely after the first protuberance has been passed.

The present invention also is of an assembly of the locking component described hereinabove, a stem of a medical device, and a catheter proximal end fitted over the stem, wherein the compression surface of the component compresses the catheter lumen wall proximally of the last or most proximal protuberance along the stem wherein the stem protuberances are preferably rounded and atraumatic to the catheter.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIGS. 1 to 4 are of the PRIOR ART, an elevation view of a venous access port of the PRIOR ART, a cross-section thereof, and isometric views of the port with a catheter lumen positioned to be urged onto a stem of the port (FIG. 3) and with the catheter lumen urged onto the stem, with a compression sleeve generally depicted along the catheter lumen to be slid over the stem to compress the lumen against the stem;

FIG. 5 is an isometric view of another venous access port having a stem with rounded protuberances;

FIGS. 6 to 8 are elevation, cross-section and end views of the locking component of the present invention; and

FIGS. 9 and 10 illustrate the use of the locking component of the present invention in locking a catheter proximal end onto the stem of the port of FIG. 5

### DETAILED DESCRIPTION OF THE INVENTION

Certain terminology is used herein for convenience only and is not to be taken as a limitation on the present invention. The terms “distal” and “proximal” refer, respectively, to directions closer to and away from the insertion tip of a catheter in an implantable catheter assembly. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

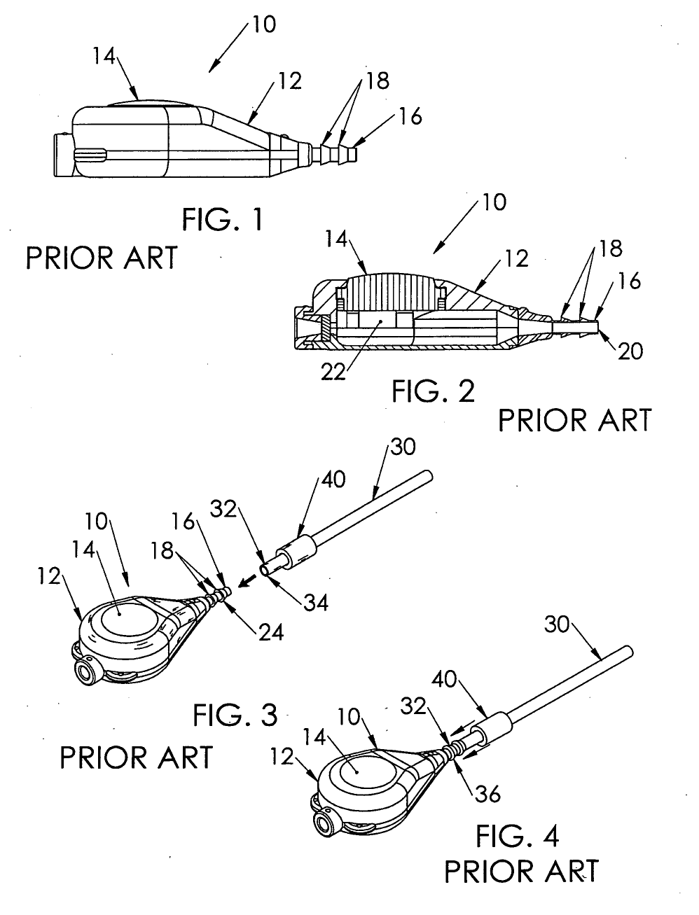

Venous access port assembly 10 of PRIOR ART FIGS. 1 to 4 includes a housing 12 and a septum 14, with a discharge port 16 extending from a distal end of the port assembly 10 to be attached securely and sealingly to the proximal end portion 32 of a catheter 30. A passageway 20 extends through the stem defining the discharge port 16 to an interior reservoir 22 that will establish fluid communication between the interior reservoir 22 and the catheter lumen 34. The stem is shown to have conventional barbed protuberances 18 onto which the catheter lumen will grip; between barbed protuberances 18 is seen a smaller diameter stem section 24. In FIG. 3, the catheter lumen is positioned and aligned with the stem 16 to be urged thereonto, with a compression sleeve 40 generally depicted along the catheter spaced from the proximal catheter end portion 32. In FIG. 4, the catheter proximal end portion has been urged onto stem 16, with expanded lumen wall sections 36 defined over the barbed protuberances; the compression sleeve may now be urged along the catheter until over and around the stem for the catheter proximal end portion to be interposed therebetween and compressed thereby onto the stem.

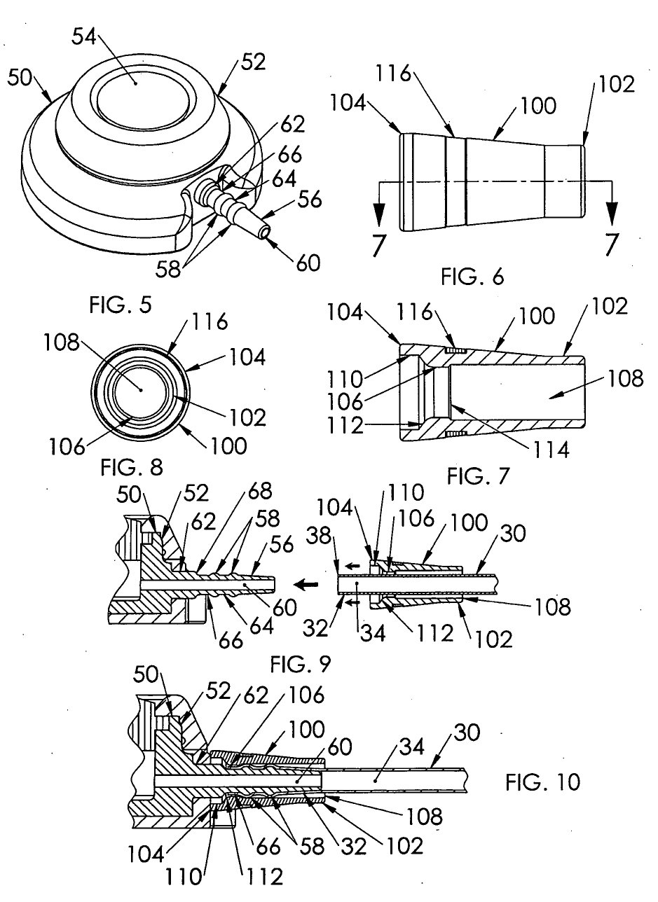

A second embodiment of medical device is depicted in FIGS. 5, 9 and 10. Venous access port assembly 50 is shown to have a second stem design 56, with a pair of spaced apart rounded annular protuberances or ribs 58 separated by a smaller diameter section 64. Port 50 and its stem 56 are of the type disclosed in U.S. patent application Ser. No. 11/801,050 filed May 7, 2007 and discussed in the Background on page 2 hereof. It is preferable for such stems to include a gradual taper distally of the most distal protuberance, in other words, its distal end portion, to facilitate insertion into the catheter proximal end. Port 50 also includes another smaller diameter section 66 portside or proximally of the second protuberance from the distal end of stem 56, that extends to a larger diameter stem section 62 adjacent its joint to port body 52, with a beveled shoulder 68 defined between stem sections 62 and 66 that is further discussed hereinbelow and seen in FIGS. 9 and 10.

Locking component 100 of the present invention is seen in FIGS. 6 to 10 an preferably comprises a one-piece sleeve body. Locking component 100 includes a distal end 102 and a proximal end 104, a channel 108 therethrough, and a compression surface 106 along the channel which has a diameter smaller than that of the remainder of the channel 108. The proximal end 104 is seen to be larger in inner and outer diameter and would be adjacent the medical device to which the catheter is to be connected, such as port 50, with the larger inner diameter region 110 being associated with larger diameter stem section 62 and adapted to receive it thereinto. A transition section 112 is provided between larger inner diameter region 110 and compression surface 106 that is angled and rounded; transition section 112 is provided such that it abuts the distal end of larger diameter stem section 62 to act as a distinct stop against further proximal movement of locking component 100 during insertion over the catheter and stem to lock the catheter onto the stem, thus assuring the accurate desired placing of the compression surface 106 portside of the final protuberance or rib of the stem (see FIG. 10) and around the smaller diameter stem section 66. The distal end of larger diameter stem section 62 also may serve as a visual indicator for the practitioner during catheter insertion over the stem, defining a fully inserted catheter position. The angled and rounded nature of transition section 112 facilitates movement of the locking component 100 over the protuberances 58 during proximal movement to minimize trauma to the catheter wall which is expanded and already stressed over the protuberances. The locking component may be metal or it may be of a plastic material such as polycarbonate.

The locking component of the present invention is conveniently placed by the practitioner onto the catheter proximal end during catheter implantation into a patient. Then, when the catheter proximal end has been urged onto the stem of the port or other medical device, the locking component can easily be slid along the catheter to surround the stem and the catheter proximal end portion interposed therebetween. As the locking component is being slid, the smaller diameter compression surface 106, constituting an interference diameter, first encounters resistance at the first protuberance 58 caused mostly by the thicknesses of the catheter lumen wall expanded thereover; the practitioner then must apply additional force to urge the component further; and the compression surface 106 passes quickly over the catheter-covered protuberance providing a tactile feel to the practitioner. Similarly, additional resistance to proximal movement of the locking component 100 occurs when the compression surface 106 encounters the second rib or protuberance 58, again requiring additional force, and again providing a tactile signal to the practitioner as the compression surface 106 passes over and beyond the second rib and is thus in its proper position over smaller diameter stem section 66 just as the transition section 112 encounters and abuts the distal end of larger diameter stem section 62, whereat the locking component 100 is finally in position adjacent to the port or medical device 50, surrounding the stem with the catheter proximal end portion 32 interposed therebetween, establishing an assured catheter locked connection to the medical device. Minimal, if any, aftereffect results to the catheter lumen wall during the brief dimensional change by the compression surface 106 at the protuberances 58.

The locking component, distally of compression surface 106 has a transition to a larger diameter channel portion 108 at a ledge 114. This ledge 114 need not be angled tapered or rounded, and can be discontinuous, which would serve to tend to resist any distal movement of the locking component along the stem with the catheter proximal end section interposed therebetween. Optionally, locking component 100 may have a marker band 116, which optionally may be barium-filled plastic or may be metal, and thereby be radiopaque to appear on imaging apparatus as a lock location indicator after catheter and port implantation. The marker band preferably is installed to be flush with the outer surface of the locking component. Furthermore, the marker band may have a particular color to be associated with a particular catheter material (e.g., polyurethane or silicone). Optionally, the locking sleeves may be slightly different dimensionally to best work with respective catheter materials or catheter wall thicknesses related thereto, and marker bands of different colors could also be used to differentiate locking component sizes to correspond with different size catheter lumens.

The locking component preferably has all outer edges and corners rounded and atraumatic. The locking component can be provided in a kit with the catheter and the venous access port or other medical device, enabling appropriately complementary geometry and dimensions. The locking component can be made of, for example, polycarbonate, but may be metal, and can be utilized with catheters that are of various materials such as silicone or polyurethane, for example.

An example of such a system using the locking component of the present invention with a stem having two protuberances and an exemplary catheter, will now be described, utilizing a stem for a silicone catheter of 9.6 F. The stem 56 has a diameter at the locking location of 0.102 in (2.590 mm), and the protuberance diameter is 0.126 in (3.200 mm) and the protuberance rounded surface has a radius of about 0.032 in (0.812 mm). The catheter lumen wall thickness on each side is 0.032 in (0.812 mm). The inner diameter of the compression surface is 0.135 in (3.429 mm). The thickness of the catheter lumen wall over the protuberances will be less because the lumen wall is expanded thereat. Thus, the inner diameter per side of the compression surface 106 is 0.0045 in (0.114 mm) greater than the diameter of each protuberance.

It is clear that the locking component can also be utilized with a stem that has one or has three (or more) protuberances that preferably are rounded and atraumatic, and still be within the spirit of the invention and the scope of the claims. Further, the locking component of the present invention could also be used with stems having the barbed protuberances.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A catheter locking component for assuredly locking a catheter end onto a stem of a medical device, where the stem has at least one protuberance therealong having a preselected maximum diameter larger than the inner diameter of the catheter's lumen for enabling the catheter end portion to grip thereonto after passing thereover, comprising:
the locking sleeve having proximal and distal ends and defining a channel of preselected diameter extending therethrough for a catheter to extend through the channel at least when the catheter has been urged onto and around the stem;
the sleeve including a compression surface along the channel having a diameter less than the preselected channel diameter when the sleeve is positioned around the stem onto which the catheter end portion has been positioned in a manner that the compression surface is axially offset proximally from the at least one protuberance.

2. The locking component of claim 1, wherein the diameter of the compression surface is incrementally larger than the maximum protuberance diameter.

3. The locking component of claim 2, wherein the diameter of the compression surface is about 0.0045 inches larger than the maximum protuberance diameter.

4. The locking component of claim 1, wherein the outer edges of the locking sleeve are rounded.

5. The locking component of claim 1, wherein a distal end of the compression sleeve is abrupt.

6. The locking component of claim 1, wherein the locking sleeve is of stiffly resilient material.

7. The locking component of claim 1, wherein the locking sleeve includes a stop surface near the proximal end of the sleeve to abut a corresponding stop surface of the stem to assure desired axial location of the compression surface relative to the proximal-most protuberance of the stem upon full insertion of the sleeve over the stem.

8. The locking component of claim 7, wherein the proximal sleeve end includes a large diameter channel portion to receive thereinto a corresponding large diameter stem portion where the corresponding stop surface of the stem is at the distal end of the large diameter stem portion to abut the stop surface at the distal end of the large diameter channel portion.

9. The locking component of claim 7, wherein the stop surface is angled distally and rounded thus facilitating, and being atraumatic to the catheter during, insertion of the sleeve over the stem.

10. The locking component of claim 9, wherein the angled and rounded stop surface is adjacent a proximal end of the compression surface.

11. An assembly of a medical device and a catheter proximal end, comprising:
a medical device having a stem having a passageway therethrough;
a catheter having at least one lumen therethrough and having a proximal end portion extending to a proximal end; and
a locking component having distal and proximal ends and a channel therethrough;
wherein the locking component includes a compression surface along the channel that has a smaller diameter than the remainder of the channel;
wherein the medical device stem has at least one protuberance extending radially outwardly therefrom to a largest outer diameter larger than the inner diameter of the catheter at the proximal end portion, and the stem has a smaller diameter section proximally of the at least one protuberance; and
wherein the locking component is adapted to be movable along the catheter and onto the catheter proximal end portion after the catheter proximal end portion has been urged onto the stem and over and beyond the at least one protuberance and along the smaller diameter section of the stem, such that the compression surface of the locking diameter compresses against the catheter end portion disposed over the smaller diameter stem section when the locking component has been fully inserted onto the stem.

12. The assembly of claim 11, wherein the at least one protuberance is rounded.

13. The assembly of claim 11, wherein the stem has two protuberances.

14. The assembly of claim 11, wherein the diameter of the compression surface is incrementally larger than the maximum protuberance diameter.

15. The assembly of claim 14, wherein the diameter of the compression surface is about 0.0045 inches larger than the maximum protuberance diameter.

16. The assembly of claim 11, wherein the outer edges of the locking component are rounded.

17. The assembly of claim 11, wherein a distal end of the compression sleeve is abrupt.

18. The assembly of claim 11, wherein the locking component is of stiffly resilient material.

19. The assembly of claim 11, wherein the locking component includes a stop surface near the proximal end of the component to abut a corresponding stop surface of the stem to assure desired axial location of the compression surface relative to the proximal-most protuberance of the stem upon full insertion of the component over the stem.

20. The assembly of claim 19, wherein the proximal component end includes a large diameter channel portion to receive thereinto a corresponding large diameter stem portion where the corresponding stop surface of the stem is at the distal end of the large diameter stem portion to abut the stop surface at the distal end of the large diameter channel portion.

21. The assembly of claim 11, wherein the stop surface is angled distally and rounded thus facilitating, and being atraumatic to the catheter during, insertion of the locking component over the stem.

22. The assembly of claim 11, wherein the angled and rounded stop surface is adjacent a proximal end of the compression surface.
